(19) United States
(12) Patent Application Publication
(10) Pub. No.: US 2006/0189966 A1
(43) Pub. Date: Aug. 24, 2006

(54) Method and system for improving vision

(75) Inventors: David Lieberman, New York, NY (US); Jonathan Grierson, Atwater, OH (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Scientific Optics, inc., New York, NY (US)

(21) Appl. No.: 10/516,833

(22) PCT Filed: Jun. 3, 2003
(86) PCT No.: PCT/US03/17633

§ 371 Date: Feb. 9, 2006

Related U.S. Application Data
(60) Provisional application No. 60/385,601, filed on Jun. 3, 2002.
(60) Provisional application No. 60/449,029, filed on Feb. 21, 2003.

(51) Int. Cl. A61F 9/008; A61B 18/18; A61B 3/10

(52) U.S. Cl. 606/12; 606/5; 351/212

(57) ABSTRACT

Methods and apparatus (610) are disclosed for diagnosing vision and improving vision in a manner which does not interfere with the natural shape of the cornea or its orientation relative to the remainder of the eye, but which changes its surface curvature appropriately to achieve the required correction of vision. The focus of the cornea (14) is adjusted so that different regions focus substantially to the same axis and preferably to substantially the same point on the axis. This can be accomplished by shaping the cornea (e.g. through ablation) or by applying an appropriate contact lens or other optical lens. In either case, correcting the central portions of the cornea should have a more significant effect on correcting focus scatter than correcting the more outward portions.

### CROSS REFERENCE OF PRIORITY APPLICATIONS

This application claims the benefit of U.S. Provisional Application No. 60/385,601, filed Jun. 3, 2003 and U.S. Provisional Application No. 60/449,029, filed Feb. 21, 2003, which are hereby incorporated by reference in their entirety.

### FIELD OF THE INVENTION

The present invention relates to a method and system for diagnosing and improving the vision of an eye.

### BACK GROUND OF THE INVENTION

Most common defects in human vision are caused by the inability of the eye to focus properly. For example, nearsightedness can be attributed to an eye which focuses forward of the retina instead of on it, farsightedness can be attributed to an eye which focuses beyond the retina, and astigmatism can be attributed to an eye which cannot produce a sharp focus, instead producing an area of blurriness. Ophthalmologists model the cornea as a portion of an ellipsoid defined by orthogonal major and minor axes. Current surgical procedures for correcting visual acuity are typically directed at increasing or decreasing the surface curvature of the cornea, while making its shape more spherical, or conforming it to an “average” ellipse, or making corrections based on wavefront analysis.

In conjunction with modern corneal procedures, such as corneal ablation surgery, for clinical applications, and for contact lens design and manufacture, high resolution cameras are used to obtain a digitized array of discrete data points on the corneal surface. One system and camera which have been available for mapping the cornea is the PAR Corneal Topography System (PAR CTS) of PAR Vision Systems. The PAR CTS maps the corneal surface topology in three-dimensional Cartesian space, i.e., along x- and y-coordinates as well as depth (Z) coordinate, and locates the “line-of-sight”, which is then used by the practitioner to plan the surgical procedure or contact lens design.

The “line-of-sight” is a straight line segment from a fixation point to the center of the entrance pupil. As described more fully in Mandell, “Locating the Corneal Sighting Center From Videokeratography,” J. Refractive Surgery, V. 11, pp. 253-259 (July/August 1995), a light ray which is directed toward a point on the entrance pupil from a point of fixation will be refracted by the cornea and aqueous humor and pass through a corresponding point on the real pupil to eventually reach the retina.

The point on the cornea at which the line-of-sight intersects the corneal surface is the “optical center” or “sighting center” of the cornea. It is the primary reference point for refractive surgery in that it usually represents the center of the area to be ablated in photorefractive keratectomy. The line-of-sight has conventionally been programmed into a laser control system to govern corneal ablation surgery. However, some surgeons prefer to use the pupillary axis as a reference line. Experienced practitioners have employed various techniques for locating the sighting center. In one technique, the angle lambda is used to calculate the position of the sighting center relative to the pupillary (“optic”) axis. See Mandell, supra, which includes a detailed discussion of the angles kappa and lambda, the disclosure of which is incorporated herein by reference as if set forth in its entirety herein.

In current corneal ablation procedures, a portion of the corneal surface or surface under a flap is ablated. The gathered elevational data is used to direct an ablation device such as a laser so that the corneal surface can be selectively ablated to more closely approximate a spherical surface of appropriate radius about the line-of-sight, (or an “average” ellipse, or a wavefront fingerprint) within the ablation zone. The use of the line-of-sight as a reference line for the procedures may reduce myopia or otherwise correct a pre-surgical dysfunction or a visual abnormality. However, a more irregularly shaped cornea may result, which may exacerbate existing astigmatism or introduce astigmatism or spherical aberration in the treated eye. This will complicate any subsequent vision correction measures that need be taken. Also, any substantial surface irregularities which are produced can cause development of scar tissue or the local accumulation of tear deposits, either of which can adversely affect vision.

Implicit in the use of the-line-of sight or the pupillary axis as a reference axis for surgical procedures is the assumption that the cornea is symmetric about an axis extending along a radius of the eye. The cornea, however, is an “asymmetrically aspheric” surface. “Aspheric” means that the radius of curvature along any corneal “meridian” is not a constant (a “meridian” could be thought of as the curve formed by the intersection of the corneal surface and a plane containing the pupillary axis). Indeed, the corneal curvature tends to flatten progressively from the geometric center to the periphery. “Asymmetric” means that the corneal meridians do not exhibit symmetry about their centers. The degree to which the cornea is aspheric and/or asymmetrical varies from patient to patient and from eye to eye within the same person.

Analysis of clinical measurements in accordance with the method disclosed in U.S. Pat. No. 5,807,381 assigned to the assignee of the present patent application, reveals that the cornea exhibits a tilt, typically a forward and downward tilt, relative to the eye. This tilt may be as great as 6° and, on the average, is between 1° and 3°. Hence, a corneal ablation procedure which utilizes the line-of-sight or pupillary axis as a reference axis tends to over-ablate some portions of the cornea and underablate other portions of the cornea. At the same time, it changes the geometric relationship between the ablated cornea and the remainder of the eye. Thus, any ablation procedure which does not take into account the tilt of the cornea is not likely to achieve the desired shaping of the cornea and may therefore be unpredictable in its effect. Similarly, a contact lens design (or any other lens used to improve vision) which does not take into account the tilt cannot achieve optimum results.

Analysis of clinical measurements in accordance with the method of U.S. Pat. No. 5,807,381 also reveals that the point on the surface of the cornea which is most distant from the reference plane of the PAR CTS (hereafter referred to as the HIGH point) is a far more effective reference point for corneal ablation than the center of the cornea or the pupillary center. Specifically, as demonstrated in U.S. Pat. No. 5,807,381 laser ablation about an axis passing through the HIGH point produces a much more regularly shaped cornea and removes less corneal material than the same operation performed about an axis close to the center of the eye, such as the pupillary axis.

Although incorporating corneal tilt and utilizing the HIGH point produced improved and more consistent results with corneal ablation surgery, there is still an excessively high degree of unpredictability. For example, analyses of clinical measurements have revealed that, in some eyes, the post-operative cornea begins to change shape a short time after corneal ablation surgery. Thus, a nearly perfectly spherical post-operative cornea of the type most commonly produced by conventional surgery, will, over time, return to an aspheric, asymmetric shape.

The present inventors believe that corneal ablation surgery has had less than optimal success and predictability, because of a parochial approach. The conventional wisdom has been to concentrate on the shape of the cornea, with the expectation that a smooth, spherical cornea (or a preconceived elliptical one) will optimize vision. However, the human eye is a complex system which includes numerous optical components besides the anterior surface of the cornea (for example, the posterior corneal surface, the crystalline lens and the aqueous humor), all of which affect vision. Also, the mechanical environment of the eye cannot be ignored. For example, recent analyses of clinical measurements reveal that the eyelids exert substantial pressure on the cornea, causing it to flatten near its upper margin and to form a depression near its lower margin. It is believed that the mechanical environment of the eye accounts, in large part, for its shape. This also explains why a perfectly spherical post-operative cornea would return to an aspherical, asymmetric shape.

In accordance with the present applicants' U.S. patent application Ser. No. 09/6,416,179 the disclosure of which is incorporated herein by reference in its entirety, corneal ablation procedures of the eye are performed in a manner which does not interfere with the natural shape of the cornea or its orientation relative to the remainder of the eye, but which changes its surface curvature appropriately to achieve the required correction of vision. Three preferred embodiments are described, which model the cornea to different degrees of accuracy. A similar approach was disclosed for selecting the shape of a lens in contact lens design.

Analysis of clinical measurements in accordance with the methods of U.S. Pat. No. 5,807,381, as refined in accordance with the present invention, raises questions about assumptions that have been made about the structure of the human cornea which are inherent in such well-known corneal analysis technologies as wave-front analysis and placido disc technology. In particular, it has been found that, unlike other optical systems, the central portion of the cornea (for example, out to a 3 mm diameter) is not optically superior to substantially greater portions of the cornea (for example, out to a 7 mm diameter) in its ability to focus. The central portion of the cornea exhibits a great deal of focus scattering. That is, different regions on the cornea do not focus to the same point on a focal axis. Indeed, they do not even focus on the axis. This difference is most pronounced in the central portion of the cornea and decreases substantially at increasing diameters from the center.

In accordance with the present invention, vision can be improved by adjusting the focus of the cornea so that different regions focus substantially to the same axis. This can be accomplished by shaping the cornea (e.g. through ablation) or by applying an appropriate corrective lens. In either case, correcting the central portions of the cornea should have a more significant effect on correcting focus scatter than correcting the more outward portions. However, it is preferred that adjustments be made to both.

### BRIEF DESCRIPTION OF THE DRAWINGS

The foregoing brief description, as well as other objects, features and advantages of the present invention will be understood more completely from the following detailed description of presently preferred embodiments, with reference being had to the accompanying drawings in which:

FIG. 1 is a block diagram illustrating a method for achieving vision correction in accordance with the present invention through either laser ablation of the cornea or an appropriately shaped contact lens;

FIG. 2 is a schematic diagram illustrating a plan view of a point cloud as obtained with a corneal image capture system;

FIG. 3 is a schematic plan view similar to FIG. 2 illustrating a plurality of splines and how they are connected through the data points of the point cloud;

FIG. 4 is a perspective view of a cornea matching surface illustrating how characterizing curves are constructed;

FIG. 5 is a diagram illustrating the axial focus scatter of a cornea at a 3 millimeter diameter.

FIG. 6 illustrates the radial focus scatter corresponding to FIG. 5;

FIG. 7 is a diagram illustrating the axial focus scatter of a cornea at a 5 millimeter diameter;

FIG. 8 illustrates the radial focus scatter corresponding to FIG. 7;

FIG. 9 is a diagram illustrating the axial focus scatter of a cornea at a 7 millimeter diameter;

FIG. 10 illustrates the radial focus scatter corresponding to FIG. 9;

FIG. 11 illustrates a method for modifying the corneal model in accordance with the present invention in order to substantially reduce focus scatter;

FIG. 12 illustrates the radius of curvature at 3 millimeters of each of the characteristic curve arcs for the corneal model, both before and after the application of the method of the present invention; and

FIG. 13 illustrates the radius of curvature of each of the characteristic curve arcs for the corneal model with a 7 millimeter diameter, both before and after the application of the method of the present invention;

FIG. 14 illustrates the radius of curvature of each of the characteristic curve arcs of the of central optical portion for a contact lens made for an eye with extreme keratoconus, both with and without orthogonalization;

FIG. 15 is a diagram similar to FIG. 14 for the peripheral optical portion of the same lens; and

FIG. 16 illustrates the variation of the radius of an actual patient's cornea as a function diameter at which the radius is measured.

### DETAILED DESCRIPTION OF THE PREFERRED EMBODIMENTS

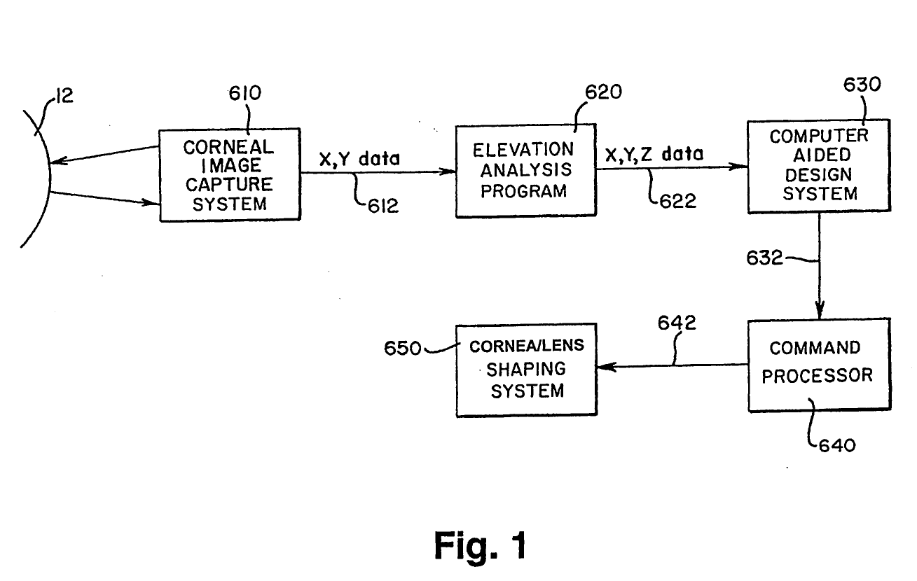

A process for achieving laser ablation of the cornea and contact lens shaping in accordance the present invention is illustrated in block diagram form in FIG. 1. The process makes use of a Corneal Image Capture System 610, an Elevation Analysis Program 620, a Computer Aided Design System 630, a Command Processor 640 and a Cornea Shaping System 650. The Corneal Image Capture System 610, in conjunction with the Elevation Analysis Program 620, generates a three dimensional topographic map of the cornea of the patient. The Computer Aided Design System 630 is used as an aid in editing or modifying the corneal topographic data, to create a surface model, and data relating to the model is sent to a Cornea Shaping System 650 via the Command Processor 640. The Command Processor 640 uses the topographic data describing the surface of the cornea to be shaped from the Computer Aided Design System 630 to generate a sequence of commands/control signals required by the Cornea/Lens Shaping System 650. The Cornea/Lens Shaping System 650 accepts, from the Command Processor 640, a sequence of commands that describe the three dimensional movements of the Cornea/Lens Shaping System (any coordinate system may be used; e.g., Cartesian, radial or spherical coordinates) to shape the cornea or machine (e.g. a lathe) manufacturing a contact lens.

The Corneal Image Capturing System 610 and the Elevation Analysis Program 620 are preferably components of the PAR® Corneal Topography System (“the PAR® System”), which is available from PAR Vision Systems. The Elevation Analysis Program 620 is a software program executed by a processor, for example an IBM™ compatible PC. Program 620 generates a third dimension element (a Z coordinate representing distance away from a reference plane inside the eye) for each of a plurality of sample points on the surface of the cornea measured by system 610. Each point is defined by its X-Y coordinates as mapped into the reference plane, and its Z coordinate is determined from brightness of the point. One method of calculating the elevation of each point, i re., the Z coordinate, is by comparing the X-Y and brightness values measured from the patient's cornea 14 with the coordinates and brightness of some reference surface with known elevation, e.g., a sphere of a known radius. The reference values can be pre-stored.

The final output of the Elevation Analysis Program 620 is the X-Y-Z coordinates for a multiplicity of sample points, known as a point cloud, on the surface of the cornea 14. It will be apparent to those skilled in the art that any method can be used that can generate X, Y, Z corneal data providing both location and elevation information for points on the corneal surface with the required accuracy. In the preferred embodiment about 1500 points are spaced in a grid pattern, as viewed in the X-Y plane, so the projections of the points into the X-Y plane are about 200 microns apart.

The X-Y-Z data output from the Elevation Analysis Program 620 can be formatted in any number of well-known machine-specific formats. In the preferred embodiment, the data are formatted in Data Exchange File (DXF) format, an industry standard format which is typically used for the inter-application transfer of data. A DXF file is an ASCII data file, which can be read by most computer aided design systems.

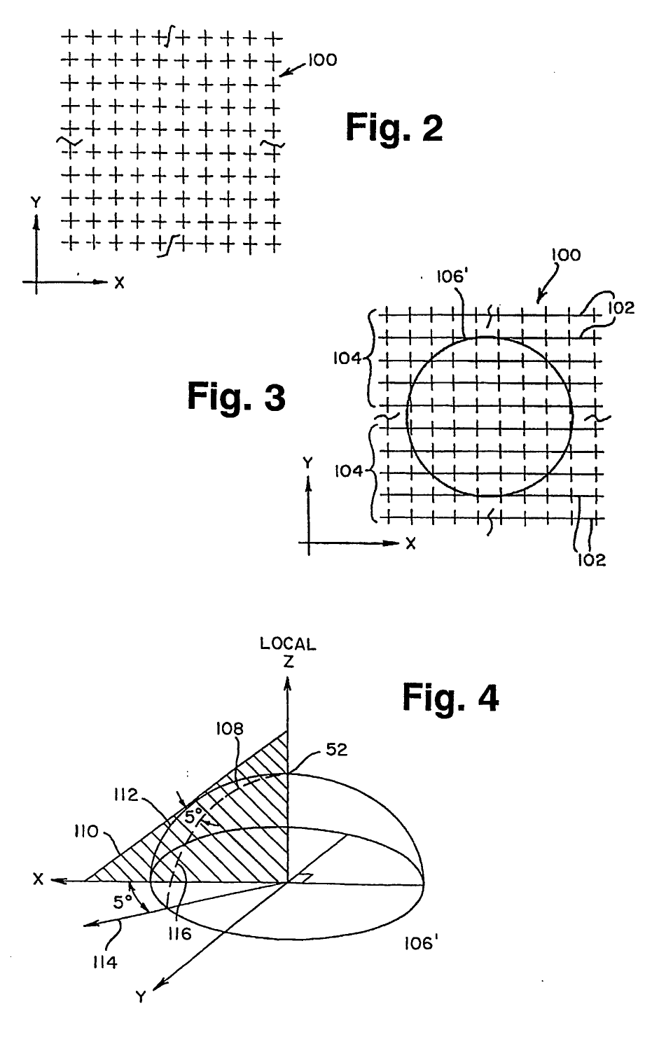

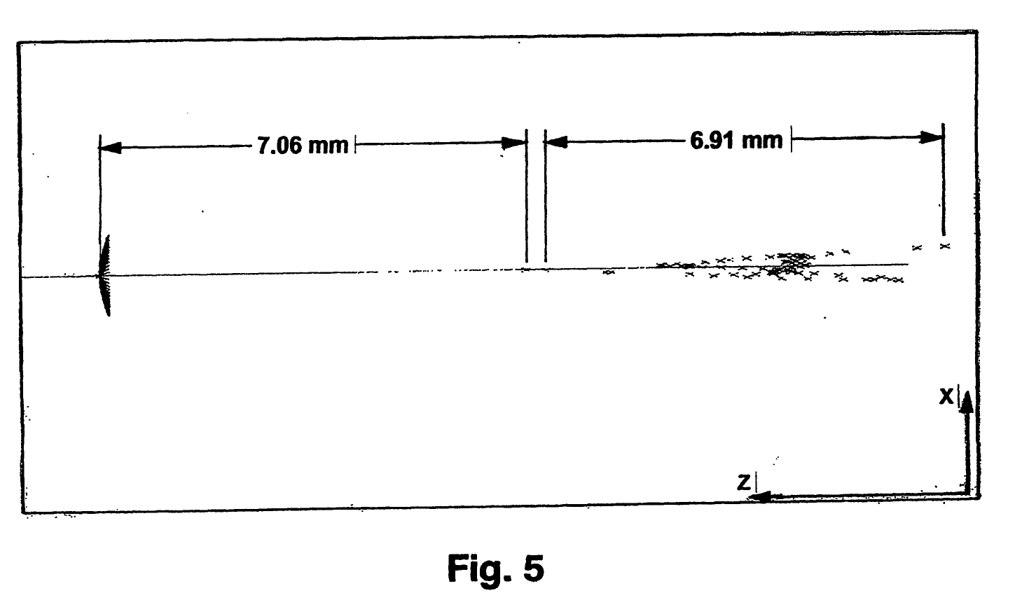

Referring now to FIGS. 2 and 3, a point cloud 100 is depicted as it would appear when viewing the reference plane along the Z-axis (i.e., as projected into the X-Y plane). Each point corresponds to a particular location on the patient's cornea. The data are usually generated from an approximately 10 mm×10 mm bounded area of the cornea, the working area. Thus, there may be as many as 50 rows of data points. A surface 108 (see FIG. 4) that models or matches the topography of the surface of the patient's cornea is generated by the computer aided design system 630 from the data points generated by the Elevation Analysis Program. In a preferred embodiment, Computer Aided Design System 630 is the Anvil5000™ program which is available from Manufacturing Consulting Services of Scottsdale, Ariz.

Cornea matching surface 108 is preferably produced by first generating a plurality of splines 102, each defined by a plurality of the data points of the point cloud 100. The generation of a spline that intersects a plurality of data points (i.e., knot points) is, per se, known to those skilled in the art and can be accomplished by the Anvil5000™ program once the input data have been entered. For more information regarding the generation of a surface model, see U.S. Pat. No. 5,807,381, the disclosure of which is incorporated herein by reference. In a preferred embodiment, the known nonuniform rational B-spline formula is used to generate the splines, but they could be generated by other well-known mathematical formulas for splines, such as the cubic spline formula or the rational uniform B-spline formula. As illustrated in FIG. 3, in a preferred embodiment, each of the splines 102 lies in a plane that is parallel to the X and Z axes and includes a row of points from the cloud 100 in FIG. 3.

Surface 108, which matches the corneal surface of the scanned eye, is then generated from splines 102. There are a number of well-known mathematical formulas that may be used to generate a surface from a plurality of splines 102. In the preferred embodiment, the well known nurb surface equation is used to generate a corneal surface from splines 102. In the embodiment, because the scanned area of the eye is approximately 10 mm×10 mm, approximately 50 splines 102 are created. As illustrated in FIG. 3, a skinned surface segment 104 is created for a small number (e.g., five) of the adjacent splines. Adjacent skinned surface segments 104 share a common border spline. Thus, about ten skinned surface segments are generated from the point cloud and are then merged together by the Anvil 5000™ program in a manner known to those skilled in the art, to produce one composite surface 108.

Neither the original data points, nor the knot points of splines 102 necessarily lie on surface 108, owing to the mathematical generation of the surface when using the nurb surface equation formula. However, the surface 108 estimates those points within a predefined tolerance.

The HIGH point on the generated corneal matching surface 108 (i.e., the point having the greatest Z value) is determined. A cylinder 106 of a predetermined diameter, is then projected onto the corneal matching surface 108 along an axis which is parallel to the Z-axis and passes through the HIGH point. Cylinder 106 preferably has a diameter of 4 mm-7 mm, typically 6 mm, and the closed contour formed by the intersection of cylinder 106 with surface 108 projects as a circle 106′ in the X-Y plane. On the matching surface 108, this contour defines the outer margin 26 of the working area of the cornea. The cornea is the most symmetric and spherical about the HIGH point and, therefore, provides the best optics at this point.

The outer margin 26 must fit within the point cloud, so that the surfaces of the cornea can be formed based on the measured corneal data. The computer aided design system 630 can then illustrate a default circle 106′ (in the X-Y plane) with respect to the point cloud, for example on a monitor screen, so that the operator can be assured that circle 106′ falls within the point cloud. Additionally, system 630 can be set up to determine if circle 106′ falls within point cloud 100 and, if it does not fall completely within point cloud 100, to alert the user to manipulate the circle (i.e., move the center point and/or change the radius of the circle) so that circle 106′ lies within the corneal data point cloud 100. In a worst case scenario, the eye should be rescanned if insufficient data is available from the scanned eye to ensure that the working area of the cornea will fit properly within the point cloud. Alternatively, the area of the point cloud can be made larger.

It is to be understood that circle 106′ is only a circle when viewed in the X-Y plane (i.e., looking along the Z-axis). Actually, the periphery 26 is approximately elliptical and lies in a plane which is tilted relative to the reference plane. A line perpendicular to this tilted plane which passes through the HIGH point will be referred to as the “LOCAL Z-AXIS” or “tilted axis”, and the tilt of the tilted plane relative to the reference plane will be considered the tilt angle of the working area of the cornea.

The cornea is about 600 μm thick. In most corneal ablation procedures, less than 100 cm depth of cornea is ablated, because there is virtually no risk of scarring with the type of lasers that are typically used. Beyond the 100 μm depth, the risk of scarring increases. For example, 120 μm depth ablation is known to cause scarring. However, there exists the possibility that the risk of scarring for deeper ablations may be reduced by drug therapy prior to or contemporaneous with the laser treatment. The magnitude of the corneal undulations is typically about fifteen to twenty microns from the crest of a hill to the trough of a valley and may be as great as about thirty microns.

The surgical procedures performed in accordance with the present invention and optical lenses manufactured in accordance with the invention will seek to correct the patient's vision in accordance , with the required corrections established in a “refraction test.” When this test is performed, the patient sits in chair which is fitted with a special device called a “phoropter”, through which the patient looks at an eye chart approximately 20 feet away. As the patient looks into the phoropter, the doctor manipulates lenses of different strengths into view and, each time, asks the patient whether the chart appears more or less clear with the particular lenses in place. In practice, the doctor is able to vary the power or diopter correction about two orthogonal axes, as well as the degree of rotation of those axes about a Z-axis along the line-of-sight. The doctor continues to modify these three parameters until he achieves the optimum vision. The results of the refraction test are usually given in the form “a, b, c°”, where “a” is the diopter correction at the first axis, “b” is the additional diopter correction required at the second, orthogonal axis, and “c°” is the angle of rotation of the first axis relative to the horizontal. This form of information is given for each eye and is immediately useful in grinding a pair of lenses for eyeglasses.

For the purposes of the present invention, it is preferred to perform a modified form of refraction test. For this modified form of refraction test, the eye doctor adjusts the phoropter at a series of equally spaced angles, say every 15° from the horizontal, and obtains the optimum refraction at each angle. Typically, the more angles that are measured, the better the results. However, since the refraction measurements can be time consuming, 15° increments, which results in the total of 12 readings seems to be a reasonable number. The manner of using the modified refraction test will be described in detail below.

There will now be described a technique for generating characterizing curves on surface 108, which will be useful below. A plane 110 is constructed which contains the LOCAL Z-AXIS (See FIG. 4). The intersection between plane 110 and surface 108 defines a first characterizing curve 112. Plane 110 is then rotated about the LOCAL Z-AXIS, for example by a 5° increment counterclockwise, as represented by line 114, where its intersection with surface 108 defines a second characterizing curve 116, which is illustrated as a dashed line in FIG. 4. This process continues at fixed rotational increments about the LOCAL Z-AXIS, for example every 5°, until plane 110 has swept 360°, to produce a complete set of characterizing curves (meridians), in this case seventy-two (360°÷5°).

Each of these characterizing curves is then estimated by a best-fit spherical (circular) arc. One manner of doing this is simply to select a circular arc which passes through three known points for each curve (e.g. the point at which it touches the contour 106′, the HIGH point, and that point which is halfway between those two points when viewed in projection along the local Z axis). Once the spherical arcs are generated, the focal point of a portion of the cornea represented by a circular arc can be estimated by the center of that arc. Techniques for locating the center of a spherical arc are well-known. The resulting set of arc centers then provides a representation of focus scattering.

For purposes of illustration, the preceding procedure was performed on the corneal model of a patient having 20/15 uncorrected visual acuity. These results are not atypical.

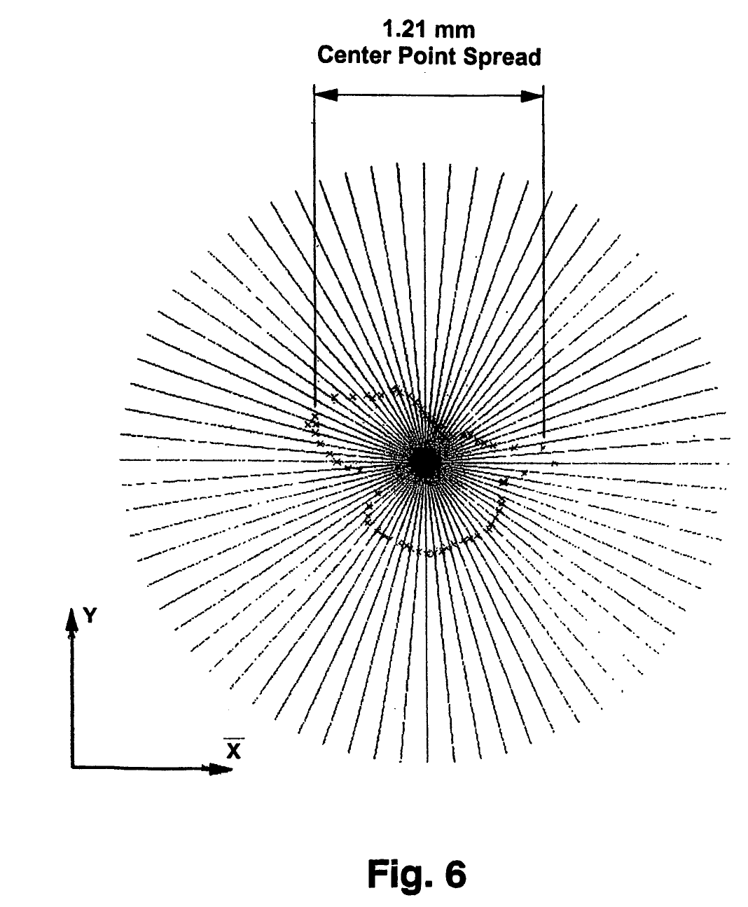

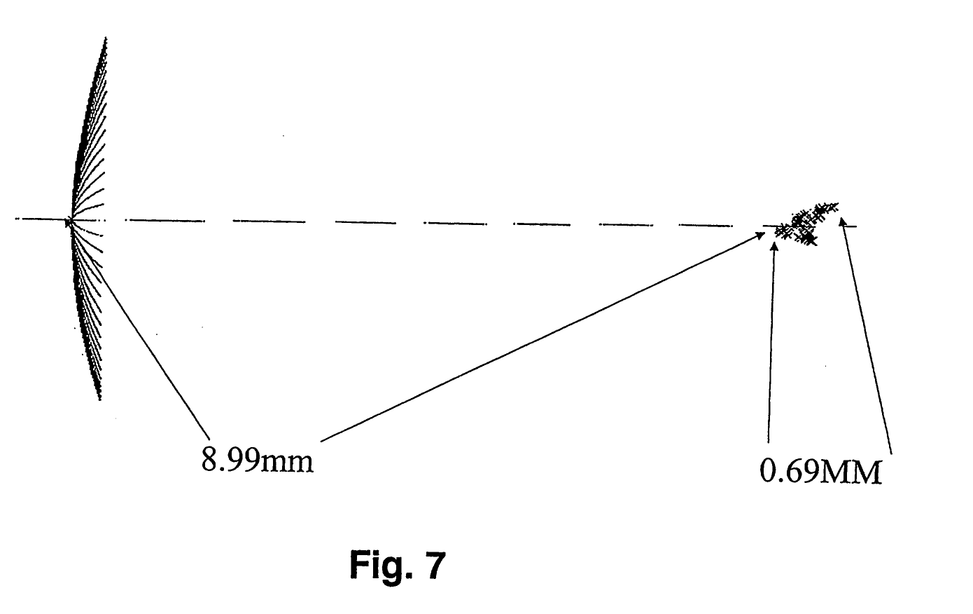

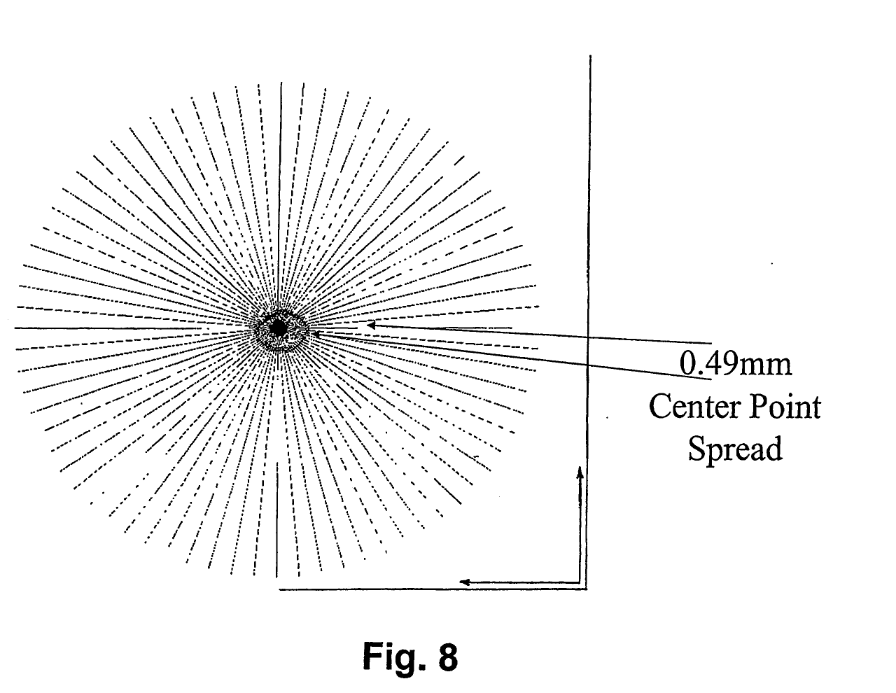

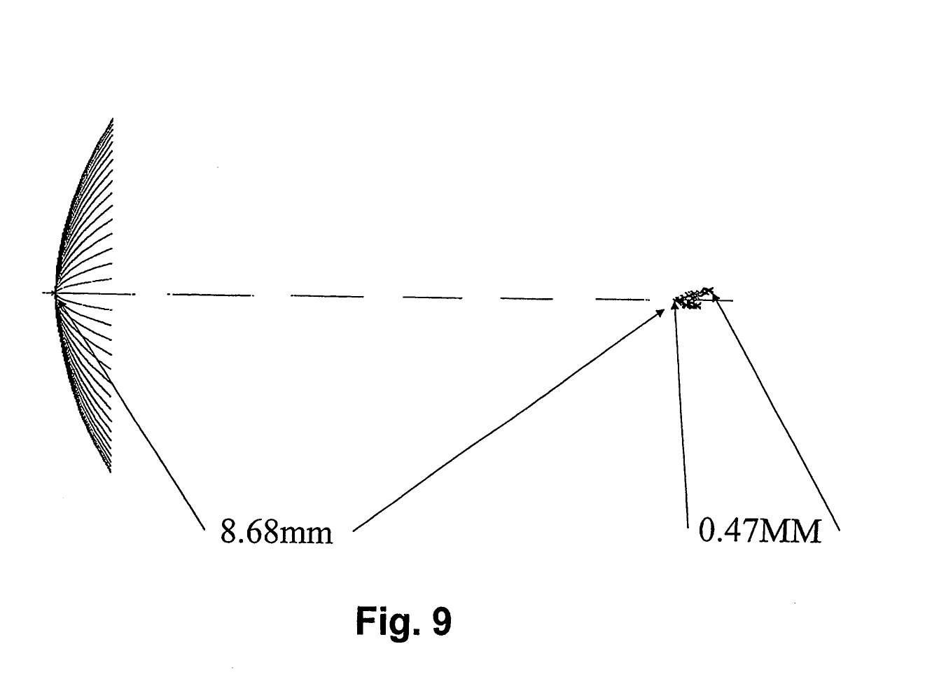

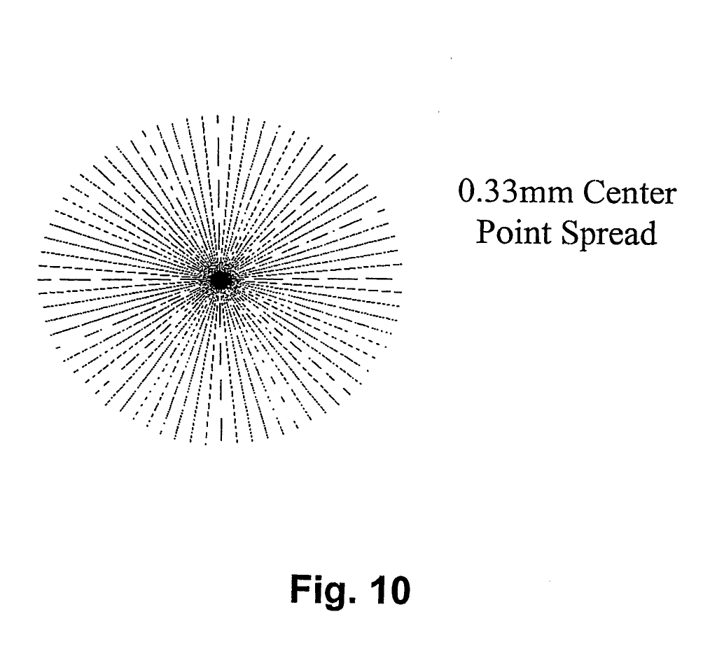

FIG. 5 is a focus scatter diagram along the LOCAL Z-AXIS for that portion of the cornea extending out to a 3.0 mm diameter. In this case, the focal points start at 7.06 mm along the LOCAL Z-AXIS and extend out an additional 6.91 mm. FIG. 6 illustrates that the radial scatter within a 3 mm diameter is 1.2 mm. Similarly, FIG. 7 illustrates that the axial focus scatter of a 5 mm diameter portion of the cornea begins at 8.99 mm and extends for an additional 1.69 mm. As shown in FIG. 8, the radial scatter of the same portion of the cornea is 0.49 mm. FIG. 9 illustrates that the axial focus scatter at 7 mm begins at 8.68 mm and extends axially for an additional 0.47 mm, whereas FIG. 10 illustrates that the corresponding radial scatter is 0.33 mm. Clearly, focus scatter is most severe in the central portion of the cornea, and decreases significantly as larger portions of the cornea are considered.

Therefore, it would clearly be desirable to reduce or eliminate the focus scatter at least in central portions of the cornea.

In accordance with the present invention, this is accomplished by “orthogonalizing” at least a portion of the cornea. The term “orthogonalizing” refers to a re-shaping of the surface model so as to piecewise re-focus the cornea towards the LOCAL Z-AXIS. The re-shaped surface model can then be applied to the cornea (e.g. through ablation) or to shape the posterior surface of a contact lens (or another type of optical lens) so as to achieve the required focus scatter correction. It has been found that orthogonalizing the cornea not only reduces radial focus scatter, but simultaneously reduces axial focus scatter substantially and produces more uniformity in the radius of curvature of the orthogonalized portion of the cornea.

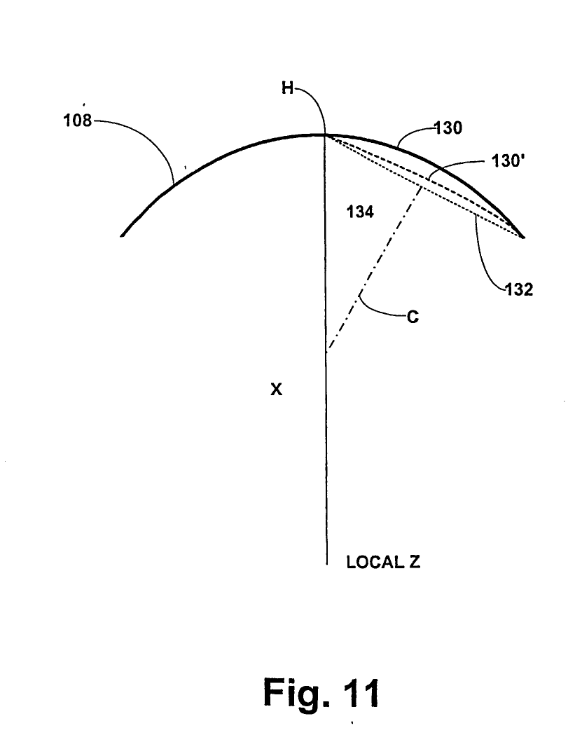

FIG. 11 illustrates the process of orthogonalization. The process is carried out on each of the arcs which represent characteristic curves, in the manner explained below. After this piecewise refocusing, the modified arcs are reassembled into a modified surface model having the re-focused characteristics.

In FIG. 11, 130 represents one of the half-meridian arcs corresponding to a characterizing curve. Arc 130 has a center point C, the 30 location of which has been exaggerated to demonstrate focus which is radially spaced from the LOCAL Z-AXIS. Orthogonalization of arc 130 begins with creating a chord 132 between the two ends of the arc. A perpendicular bisector 134 of chord 132 may be constructed, and it will pass through point C and intersect the LOCAL Z-AXIS at a point X. Using the distance of point X from point H (the HIGH point) as a radius, a new arc 130′ can now be drawn between the two end points of arc 130. Arch 130′ will be focused on the LOCAL Z-AXIS and will have a larger radius of curvature than arc 130.

At this point, arc 130′ could be accepted as an arc defining the modified surface model 108′. However, it would be desirable to avoid too great a change in the thickness of the cornea. Accordingly, a certain threshold C is defined (for example 0.0075 mm), and if any portion of arc 130′ is more than a distance e inside or outside the surface 108, arch 130′ is not accepted for use in the modified surface model. Instead, point x can be moved up or down on the LOCAL Z-AXIS (depending upon which direction arch 130′ needs to be moved) by half the excess over e. Arc 130′ can then be re-drawn and re-tested against ε. This readjustment and testing continues until an acceptable arc 130′ has been found. Then, the next arc is orthogonalized. After all of the arcs are orthogonalized, a new surface model 108′ is created based upon all of the arcs.

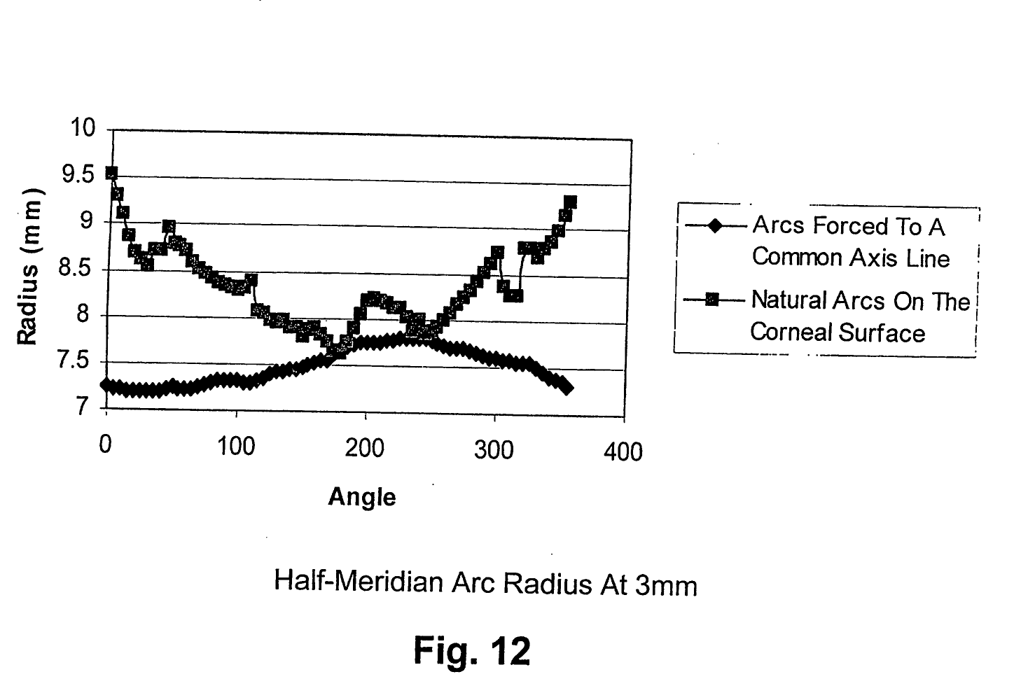

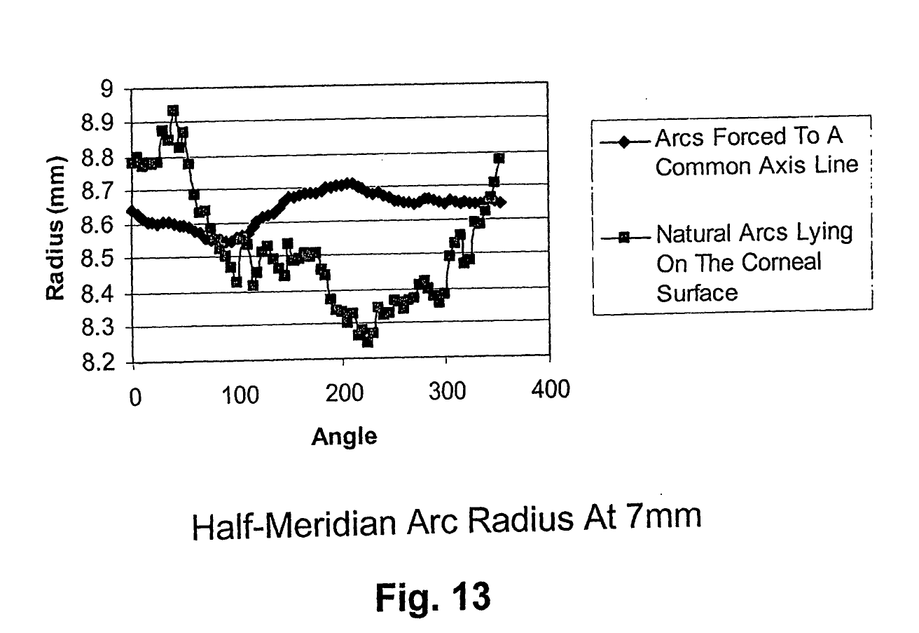

FIGS. 12 and 13 are graphs illustrating the radius of curvature at each of the 72 arc locations, both before and after orthogonalization. FIG. 12 relates to a corneal section of 3 mm diameter and FIG. 13 relates to a, section of 7 mm diameter. As can be seen, in each instance, the variation in the radius of curvature of the half-meridian arcs is substantially reduced by orthogonalization.

When the present invention is used with respect to a contact lens, the lens preferably has the structure of lens 10 illustrated in FIGS. 7A & 7B of U.S. Pat. No. 5,880,809, the disclosure of which is incorporated herein by reference in its entirety. Contact lens 10 preferably has an inner optical portion 36, a peripheral optical portion 38, and an outermost peripheral portion 34, the posterior surface of which asymmetrically and aspherically matches a corresponding portion of the cornea. This corresponding portion of the cornea lies under the outermost portion of the lens when the lens is worn in the wearer's eye. In accordance with the present invention, the inner optical portion 36 and the peripheral optical portion 38 are orthogonalized independently. That is, inner optical portion is orthogonalized as described above, and the corresponding portion of the corneal surface model is modified. The same procedure is then followed by constructing spherical arcs along half-meridians lying in the peripheral optical portion 38, following which that portion of the corneal model is modified. As explained above, in a contact lens, the modified corneal model is used to shape the posterior of the contact lens. The anterior surface of the contact lens is shaped to obtain the required visual correction for the patient, as described in U.S. Pat. No. 5,880,809.

As an example, of the improvement in vision obtainable with the present invention, the case can be considered of a patient with a severe keratoconic eye. As is common with this disorder, the patient was seeing three images in this eye: a central image and two peripheral images. When the patient was fitted with spectacles, the central image could be corrected to, at best, 20/200, but the patient still saw three images. The patient was unable to use a conventional contact lens, because such lenses fell out of the keratoconic eye. When the patient was fitted with a lens as shown in FIGS. 7A & 7B of U.S. Pat. No. 5,880,809, the lens was retained in the eye. The central image could be corrected to, at best, 20/40, but the patient still saw three images. When the patient was fitted with a contact lens as illustrated in FIGS. 7A & 7B of U.S. Pat. No. 5,880,809 with the inner optical portion 36 and the peripheral optical portion 38 independently orthogonalized, the patient saw a single image and his vision was correctable to 20/30.

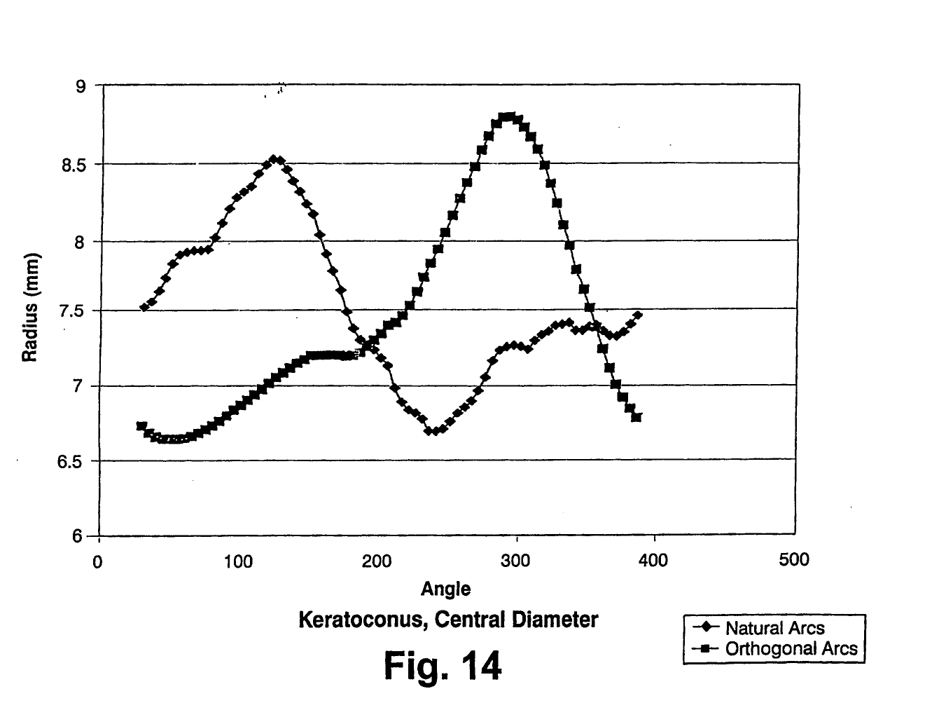

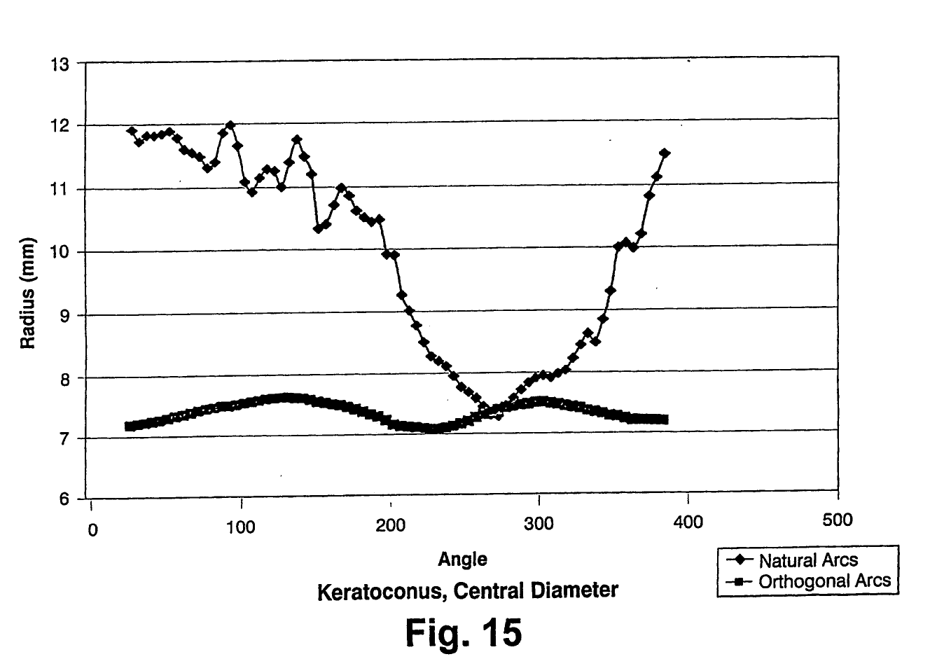

FIG. 14 illustrates the radius of curvature of each of the half-meridian arcs of the central optical portion for the keratoconic eye, both with and without orthogonalization. FIG. 15 is a similar diagram for the peripheral optical portion. As can be seen in FIG. 15, orthogonalization made the radius of curvature over the peripheral optical portion substantially uniform. Apparently, this eliminated the peripheral images that the patient was seeing.

The keratoconic eye benefitted dramatically from orthogonalization in the peripheral optical portion. It is contemplated that contact lenses in accordance with the present invention need not be limited to two optical zones. That is, the lens could have a posterior surface with a central optical zone and two or more peripheral optical zones, which are progressively further from the center, all of the optical zones being orthogonalized independently.

As far as patients with less severe conditions are concerned, all have discovered some favorable change in visual perception when using orthogonalized contact lenses. The most common improvements reported beyond the normal correction of acuity are increased depth perception and increased color perception. Also, the symptoms of presbyopia are greatly reduced or eliminated. That is, presbyopic patients may be fitted with a contact lens that does not have components which focus at different distances, and they will not require reading glasses. This is not limited to patients with small refractive errors.

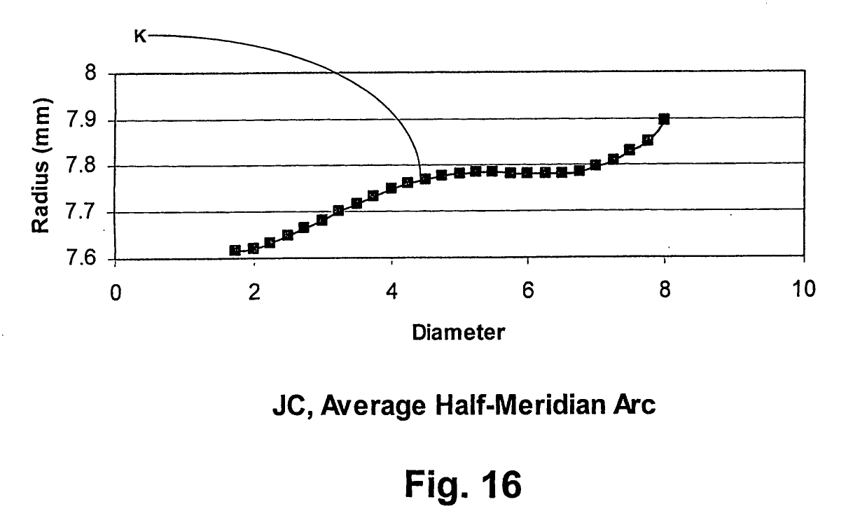

FIG. 16 illustrates how a cornea in an eye of an actual patient varies in curvature (radius) at different diameters (distance from the LOCAL Z-AXIS). This curve exhibits a slight “knee”, K, representing a relatively rapid change in curvature. Using surface model analysis, it has been found that this knee, although its location is cornea specific, is present in every eye, but becomes more pronounced as visual acuity decreases. It has also been found that if a lens is orthogonalized to a diameter less than that at which the knee occurs (e.g. the central zone ends inward of the knee), multiple images and ghosting will result. In most eyes, the knee occurs within approximately a 4.5 mm diameter. So, as a rule of thumb, this disastrous defect can be avoided by assuring that the central zone extends beyond approximately a 4.5 mm diameter.

As has been explained above, the orthogonalization process is applicable to corneal ablation procedures. Prior to the procedure, a corrected corneal surface model is generated, which is shaped to provide the correction refraction established by an eye test (as described in the patents cited above), and it is orthogonalized. The corrected corneal surface model is then registered with the unmodified corneal surface model, and it is moved towards the unmodified surface until the corrected surface just contacts the unmodified surface. If the point of initial contact is at the center of the corrected surface, it is moved toward the uncorrected surface until the periphery of the corrected surface just contacts the uncorrected surface. If the point of initial contact is at the periphery of the corrected surface, it is moved toward the uncorrected surface until the center of the corrected surface just contacts the uncorrected surface. The corrected surface will then be displaced so that it is, at least partially, inside the cornea, and the cornea is ablated until the displaced corrected surface becomes its new surface.

This procedure can be expected to reduce substantially the amount of material removed from the cornea, in comparison to all prior ablation techniques.

Although preferred embodiments of the invention have been disclosed for illustrative purposes, those skilled in the art will appreciate that many additions, modifications, and substitutions are possible without departing from the scope and spirit of the invention. For example, the present invention is applicable not only to corneal ablation and contact lenses, but to any other kind of lens, including cataract, phakic, intraoccular, intracorneal and spectacle lenses.

1. In a method for improving or planning the improvement of the vision of an eye, the steps of, on a surface model of the cornea of the eye, determining points of focus for different locations on the surface model and modifying the model so as to shift points of focus to a predefined reference axis, without forcing them to a common point the modified model representing a desired restructuring of the cornea.

2. The method of claim 1 wherein said modifying step is representative of effectively re-shaping the cornea by one of physically changing its shape and applying to the eye an optical lens intended to correct refractive error.

3. The method of claim 2 wherein physical changing comprises an intended corneal ablation on the cornea of the eye.

4. The method of claim 2 wherein the optical lens is one of a contact lens, a cataract lens, a phakic lens an intraocular lens, an intracorneal lens and a spectacle lens.

5. The method of any preceding claim wherein the reference axis passes through the HIGH point.

6. The method of any preceding claim wherein the reference axis is the LOCAL Z-AXIS.

7. The method of any preceding claim wherein the surface of the cornea is modeled in terms of a central cap-shaped portion and at least one peripheral band portion radially outward of the cap-shaped portion.

8. The method of claim 7 wherein there are a plurality of band portions successively radially outward of each other.

9. The method of any one of claims 7 or 8 wherein a periphery of the cap-shaped portion is at least approximately 4.5 millimeters away from the reference axis.

10. The method of any preceding claim performed with the aid of computer program which produces the surface model of the cornea, which closely represents at least a portion of the surface of a cornea in three dimensions as a smooth, free-form surface, the modifying step comprising changing the shape of at least a portion of the model to produce a modified surface model.

11. The method of claim 10, wherein the modifying step corresponds to one of conforming the shape of at least a portion of the cornea to the modified surface model, and conforming the shape of at least a portion of a surface of an optical lens to the modified surface model.

12. The method of any one of claims 10 or 11 wherein a central cap-shaped portion is modeled on the original surface model as a series of arcs, rotationally spaced about the reference axis and conforming to the surface model, said multiple locations being selected arcs extending between the reference axis and the periphery of the cap-shaped portion, an arc being refocused by: locating the point X at which the perpendicular bisector of a chord between the ends of the arc intersects the reference axis; the distance between point X and the intersection of the reference axis with the surface model being used as a radius to scribe, from point X, a modified arc between the two ends of the arc; and smoothly joining modified arcs to define the modified surface model.

13. The method of any one of claims 10-12, wherein a band portion, radially outward of a central cap-shaped portion, is modeled on the original surface model as a series of arcs rotationally spaced about the reference axis and conforming to the surface model, said multiple locations being selected arcs extending between peripheries of a band portion, an arc being refocused by: locating the point X at which the perpendicular bisector of a chord between the ends of the arc intersects the reference axis; the distance between point X and the intersection of the reference axis with the surface model being used as a radius to scribe, from point X, a modified arc between the two ends of the arc; and smoothly joining modified arcs to define the modified surface model.

14. The method claim 12 or 13 further comprising the step of, after generating a modified arc, measuring the maximum distance of that arc from the corresponding original arc and, if that distance exceeds a threshold value, moving the point X along the reference axis and scribing a new modified arc from moved point X which does not exceed the threshold value.

15. The method of any one of claims 10-14 further comprising the steps of:
orienting the modified surface model in correspondence with the unmodified surface model;
moving the two models together until they just make contact;
if the point of initial contact is near the center of the modified surface model, moving the two models together further until the periphery of the modified surface model just contacts the original surface model.

16. An optical lens for improving the vision of an eye, the lens comprising areas of focus on a surface thereof corresponding to different locations on the corneal surface of the eye, each area of focus being shaped to shift the focus of the corresponding location of the cornea to a predefined reference axis in the eye, without forcing the focus of each area to a common point.

17. The lens of claim 16 wherein the lens comprises one of a cataract lens, a phakic lens an intraoccular lens, an intracorneal lens and a spectacle lens.

18. The lens of any one of claims 16 or 17 wherein the reference axis passes through the HIGH point.

19. The lens of any one of claims 16-18 wherein the reference axis is the LOCALZ-AXIS.

20. The lens of any one of claims 16-19 wherein the surface of the lens is constructed as a central cap-shaped portion and at least one peripheral band portion radially outward, with respect to said referenced axis, of the cap-shaped portion.

21. The lens of claim 20 comprising a plurality of band portions successively radially outward of each other.

22. The lens of any one of claims 20 or 21 wherein a periphery of the cap-shaped portion is at least approximately 4.5 millimeters away from the reference axis.

23. The lens of any one of claims 16-23 designed with the aid of computer program which produces a surface model of the cornea which closely represents at least a portion of the surface of a cornea in three dimensions as a smooth, free-form surface, the model being modified in shape at each corresponding location at least a portion of the lens conforming in shape to the modified surface model.

24. In a system for improving the vision of an eye by effectively reshaping the cornea by one of controlling physically changing the shape of the cornea and controlling the shape of a lens to be applied to the eye to correct its refractive error, a controller which controls said reshaping so as to shift points of focus for different locations on the surface of the cornea to a predefined reference axis, without forcing the focus of each area to a common point.

25. The system of claim 24 wherein the lens comprises one of a cataract lens, a phakic lens anintraoccular lens, anintracorneal lens and a spectacle lens.

26. The system of any one of claims 24 or 25 wherein the controller causes reference axis to pass through the HIGH point.

27. The system of any one of claims 24-26 wherein the controller causes the reference axis to be substantially coincident with the LOCAL Z-AXIS.

28. The system of any one of claims 24-27 wherein the controller causes the surface of the lens to be constructed as a central cap-shaped portion and at least one peripheral band portion radially outward, with respect to said referenced axis, of the cap-shaped portion.

29. The system of claim 28 wherein the controller causes the lens surface to include a plurality of band portions successively radially outward of each other.

30. The system of any one of claims 28 or 29 wherein the controller causes a periphery of the cap-shaped portion to be at least approximately 4.5 millimeters away from the reference axis.

31. The system of any one of claims 28-30 wherein the controller makes use of computer program which produces a surface model of the cornea which closely represents at least a portion of the surface of the cornea in three dimensions as a smooth, free-form surface, the controller causing the model to be modified in shape at each corresponding location so that at least a portion of the lens conforms in shape to the modified surface model.

32. A method for analyzing the cornea of an eye, including analysis for improving or planning the improvement of vision, said method making use of a surface model of the cornea of the eye which closely represents at least a portion of the surface of the cornea in three dimensions as defined with a predefined reference axis, said method comprising the steps of:
a) creating a characterizing curve of the surface model as the intersection between the surface model and a plane containing the reference axis, the characterizing curve being between the axis and a predefined peripheral limit of the surface model;
b) estimating the characterizing curve by a close fitting, substantially circular characterizing arc;
c) rotating the plane about the reference axis and repeating steps a) and b) to create and additional arc, steps a), b) and c) being repeated to created a predetermined number of characterizing arcs representing said surface model; and
d) creating a display of arc radius versus angular position of the plane, as a characterization of the surface model.

33. The method of claim 32, wherein the portion of the cornea is generally cap shaped.

34. The method of claim 32, wherein the portion of the cornea is a peripheral band which, with respect to the reference axis, is outward of generally cap shaped portion.

35. The method of any one of claims 32-34 performed with the aid of computer program which produces the surface model of the cornea to closely represent at least the portion of the surface of the cornea in three dimensions as a smooth, free-form surface.
